(19) United States
(12) Patent Application Publication
(10) Pub. No.: US 2011/0146671 A1
(43) Pub. Date: Jun. 23, 2011

(54) PHARMACEUTICAL COMPOSITIONS AND METHODS OF DELIVERING THE SAME

(76) Inventors: James Zhou Liu, Downingtown, PA (US); Lixin Lilly Zhang, Downingtown, PA (US)

(21) Appl. No.: 11/944,616

(22) Filed: Nov. 24, 2007

Related U.S. Application Data
(60) Provisional application No. 60/868,214, filed on Dec. 1, 2006.

(51) Int. Cl. A61M 11/00; A61K 36/886; A61P 11/00; A61P 29/00; A61M 31/00

(52) U.S. Cl. 128/200.23; 424/744; 604/213; 604/514; 604/191

(57) ABSTRACT

The present invention provides a pharmaceutical composition comprising a chemical compound or a herb extract or the combination having at least one of the activities of antiviral, antibacterial, antifungal, anti-inflammatory, anti-allergy, anti-cancer, promoting membrane healing or immunomodulating, for the treatment and prevention of a disease of a mammal. The present invention also provides a system for delivering the composition to the hard-to-reach sinuses of a mammal to reduce the load of the pathogen, to reduce the amount of inflammatory mediators, to heal pathological changes, and collectively to treat a disease of a mammal, such as allergic fungal rhinosinusitis. The invented method of treating a human disease reduces infectious and inflammatory agents without causing antibiotic resistance or adverse event. The present invention additionally provides a kit for collecting the nasal wash specimen to analyze how much drug is administered into sinonasal cavities or to determine the load of microbes in rhino-sinuses.

### TECHNICAL FIELD OF THE INVENTION

The present invention relates to a pharmaceutical composition comprising a chemical compound or a herb extract or the combination having at least one of the activities of antiviral, antibacterial, antifungal, anti-inflammatory, anti-allergy, anti-cancer, promoting membrane healing or immunomodulating, for the treatment and prevention of a disease of a mammal. The present invention also provides a system for delivering the composition to the hard-to-reach sinuses of a mammal to reduce the load of the pathogen, to reduce the amount of inflammatory mediators, to heal pathological changes, and collectively to treat a disease of a mammal, such as allergic fungal rhinosinusitis. The new method of treating a human disease not only reduces infectious and inflammatory agents, also not cause antibiotic resistance, neither any adverse event. The present invention additionally provides a kit for collecting the nasal wash specimen to analyze how much drug is administered into sinonasal cavities or to determine the status of microbes in rhino-sinuses.

### BACKGROUND OF THE INVENTION

Modern antibiotics gradually become less effective due to multiple drug resistance developed by many microbes. A pharmaceutical formulation with a single chemical drug is hard to achieve therapeutic efficacy for a variety of conditions, such as chronic allergic fungal rhinosinusitis. Therefore, an alternative method must be developed to treat human diseases. The present invention is to provide a pharmaceutical composition containing a number of active components to have a combinational effect in treating human diseases.

Nasal drug delivery generally is safer and easier by the user than injection to administer a therapeutic agent in a variety of conditions. Recently, nasal drug administration has been used as an alternative route for the local and systemic availability of drugs restricted to administration intravenously, orally or other routes. This is due to the fact that sinonasal cavities have a large surface area, porous endothelial membrane, high total blood flow, the avoidance of first-pass metabolism, and ready accessibility. The nasal administration of drugs, including numerous compounds, peptide and protein drugs, for local and systemic medication has been widely investigated in recent years. However, if drugs are only applied onto the surface of nasal passage, not into sinuses, are cleared rapidly from the nasal cavity. Generally, drugs administered with a spray device, or delivered in aerosol with an aerosol generating mean, are hardly entering into sinuses. Hence, administration of drugs to sinuses is very challenging and needs a new solution. This invention is to meet this special need.

According to The American Academy of Otolaryngology-Head and Neck Surgery (AAO-HNS), more than 37 million Americans suffer from at least one episode of sinusitis each year (http://www.entnet.org/healthinfo/sinus/sinus_questions.cfm). The prevalence of sinusitis has soared in the last decade possibly due to increased pollution, urban sprawl, and the increased number of those microorganisms highly resistant to antibiotics, or super bugs. A recent estimate suggests that 70 million people in America suffer from nasal disorders and a similar number in Europe, making it one of the most common chronic illnesses. The treatment of rhinosinusitis is poorly resolved by current nasal delivery systems because the drug does not adequately reach the upper posterior of the nasal passage, neither sinuses. The newly invented device with a continual positive pressure (CPP) technology is to enable the therapeutic solution enter into the nose, then into the posterior nasal passage beyond the nasal valve, and finally into sinuses. At the same time the therapeutic solution dissolve infectious and inflammatory materials in nasal and sinus cavities and remove them from those hard to reach areas.

Allergic fungal rhinosinusitis is a hard to treat disease. A Mayo study reported that fungal growth was found in nasal washings from the sinuses in 96% of patients with chronic sinusitis (Ponikau et al. The diagnosis and incidence of allergic fungal sinusitis. Mayo Clin. Proc. 74:877-884, 1999). Normal controls had almost as much growth, the difference being that those patients with chronic sinusitis had eosinophils, a type of white blood cell involved in allergic and other reactions, which had become activated. As a result of the activation, the eosinophils released a product called major basic protein (MBP) into the mucus which attacks and kills the fungus but is very irritating to the lining of the sinuses. It is believed that MBP injures the lining of the sinuses and allows the bacteria to proliferate. A number of fungi and bacteria have been detected in sinuses of the patients with chronic rhinosinusitis (Nigro et al. Microbiology of the maxillary and ethmoid sinuses in patients with chronic rhinosinusitis submitted to functional endoscopic sinus surgery. Rev Bras Otorrinolaringol 2006; 72(2):217-222.). Based on the description by Ryan, Matthew, Marple, Bradley (Current Opinion in Otolaryngology & Head & Neck Surgery. 15(1):18-22, February 2007.), most patients have detectable fungi, fungal-specific IgE in their allergic mucin. Elevated levels of fungal-specific IgG3 is also a consistent finding in patients with allergic fungal rhinosinusitis and eosinophilic mucin chronic rhinosinusitis. Antifungal treatment is still considered a treatment option, but a combinational therapy could be more effective. The present invention is to remove all those pathogenic factors: fungi, eosinophils, IgE, IgG3, MBP, and bacteria from nasal and sinus cavities while anti-infection and anti-inflammation agents will be delivered into nasal passage and sinuses.

Influenza viruses A, B and C, including bird flu viruses, respiratory syncytial viruses A and B, SARS virus, parainfluenza viruses and the like are common causes for respiratory tract infections in humans. Presently, it is lack of effective drugs, vaccines or other means to treat or prevent these viral infections prior to identification of the pathogens. Human adults and school children are constantly exposed to these infectious agents at work and/or at school, and may also be carriers of these infectious agents to and from their home. A practice of sinonasal cleaning can reduce the microbial load of those tissues and decrease the chance of spreading these disease-causing microorganisms at home, school and working place.

Many disease-causing agents exist in the sinonasal cavities. The shedding of communicable infectious microorganisms in the sinonasal cavities causes the spread of the disease from the upper respiratory tract to the lower respiratory tract of the carrier. Shedding also causes the spread of these infectious agents to other people through sneezing and/or coughing (Hall, C. B., Douglas, R. J., in an article entitled: “Quantitative shedding patterns of respiratory syncytial virus in infants”, Journal of Infectious Diseases, 132: 151-156, 1975; and Hall, C. B., Geiman, J. M., Breese, B. B., and Douglas, R. J., in an article entitled “Parainfluenza virus infections in children: Correlation of shedding with clinical manifestations”, Journal of Pediatrics, 91: 194-198, 1977). For most viral infections, antibiotic prevention and/or treatment are generally ineffective. It would thus be beneficial to have a practical system and/or method to remove these disease-causing agents from the sinonasal cavities. In a manner similar to brushing teeth, sinonasal delivery of the therapeutic liquid will remove those pathogenic factors from sinonasal cavities, hence, not chance for those microbes to develop drug resistance. More importantly, the new treatment will have no side effect. The general population has a number of healthy benefits by using a new method which is provided in this invention.

Nasal washes, nasopharyngeal swabbing and nasopharyngeal aspiration have been used to obtain specimens from patients for the determination of microbial pathogens (Hall, C. B., Douglas, R. J., in an article entitled: “Clinically useful method for the isolation of respiratory syncytial virus”, Journal of Infectious Diseases, 131: 1-5, 1975). However, these procedures and devices were used only to obtain samples and are not effective in removing the infectious agents from the nasal and nasopharyngeal cavities of a human.

Ephedrine nasal washes have been used in the treatment of sinusitis and other nasal and paranasal symptoms and allergic rhinitis, (Shaikh, W. A., in the Journal of Allergy Clinical Immunology, Vol. 96, No. 5, part 1: 597-600, 1995). The Shaikh procedure uses a 1% ephedrine hydrochloride solution in a normal saline solution and a Higginson's rubber syringe. After the rubber syringe is filled with the wash solution, the nozzle of the syringe is introduced into one nostril and the bulb of the syringe is pressed to push the fluid into the nasal cavity. As described by the author, most of the fluid exited from the same nostril, but some fluid exited through the other side of the nose after passing through the nasopharynx. This procedure was performed once every forty-eight (48) hours for a four (4) week period and caused a significant improvement in symptom scores and peak nasal inspiratory flow rates in patients with perennial allergic rhinitis as compared to those treated with a placebo wash (normal saline only). This procedure, however, has the following disadvantages: (1) ephedrine was the key factor for the effectiveness of this procedure, but this chemical is not suitable for use by the general public on a daily basis; (2) this procedure was performed only on patients with perennial allergic rhinitis; (3) this procedure was mainly washing of the nasal cavity, the sinus and nasopharyngeal cavities were barely cleansed; (4) the apparatus used was clumsy and uncomfortable to use; and (5) this procedure was performed once every forty-eight (48) hours, which is not frequent enough to remove harmful materials from the sinonasal cavities on a daily basis. The Shaikh procedure would permit the infectious microorganisms to be brought into and spread around at home, office, school, or day care center. Therefore, there is a need to develop a generally acceptable and more effective sinonasal cleaning system.

Sinonasal cavities are common places for holding environmental allergens, such as pollen, fungal spores, animal body-originated dustings and volatile chemicals. When inhaled, these harmful agents cause allergic reactions and other ill consequences. Sinonasal secretions combining with environmental particles form big matters (solidified mucous) in the nasal cavities. These big matters narrow the airway and add to nasal obstruction. The present invention aims to easily and effectively remove those harmful agents from the sinonasal cavities and to prevent the formation of and remove the big matters in the nasal cavity.

The human body is the natural host for many kinds of pathogenic microorganisms. Sinus and nasopharyngeal mucous is one of the prominent places of viral shedding. These pathogens include, but are not limited to, influenza viruses, respiratory syncytial viruses and the like. The nasopharyngeal shedding of these pathogens is the major cause of person-to-person transmission. One skilled in the art will appreciate that those communicable pathogenic microorganisms present in the nasal, sinus and nasopharyngeal cavities will be decreased in quantity after the cavities have been cleaned. After sinonasal cleaning, these infectious agents will be less likely to spread horizontally to non-carriers and/or vertically to the lower respiratory tract of the carrier.

Environmental pathogens can be encountered by inhalation. Legionella pneumophila, the causative agent of Legionnaire's disease presents in aerosols. It is generated from air conditioning cooling towers, cold water taps, showers and other water systems. Depending upon wind speed, Legionella pneumophila, in these aerosols, can be carried up to 500 meters and infect a large number of individuals. Promptly removing these aerosols from the nasal, sinus and nasopharyngeal cavities will greatly reduce the incidence of infections.

Several methods have been reported to be useful in cleaning nasal cavities. Grossan invented a nasal irrigation system (NASAL IRRIGATION SYSTEM, U.S. Pat. No. 3,047,145, issued Nov. 12, 1974) which provided for an isotonic saline solution under pressure flowing into one nostril, passing through the nasolacrimal duct, where the solution passes, sequentially, into the ostia of the frontal sinus, the ethnoids, the maxillary and the sphenoid. The solution then moves past the outlet of the Eustachian tube and then through the nasopharynx to the upper posterior portion of the other nostril and outwardly there through, passing the same ducts and ostia, in reverse sequence, before being discharged from the second nostril. This system has numerous and serious shortcomings. First, the machine needs electricity, bulky and heavy so it is not convenient for home and traveling use. Secondly, the machine is difficult to clean and has potential water-electric hazard. Thirdly, it requires big volume of liquid, not suitable for nasal drug delivery. Therefore, the reported nasal irrigation system of Grossan cannot be widely used by the general public for cleaning nasal and nasopharyngeal cavities.

A method of administrating a pharmacological solution into the nasal cavity of a patient was described by Lofstedt (METHOD FOR DRUG ADMINISTRATION, U.S. Pat. No. 5,116,311, issued May 26, 1992). Their device has a compressible container, which not only forces the solution flow into the nasal cavity, but also aspirates the solution with nasal secretions and other contaminants back into the container. It is not an ideal device for nasal drug administration.

Pena invented a device for treating infections of the nasal fossae (DEVICE FOR CIRCULATING TREATING FLUID THROUGH THE NASAL FOSSAE, U.S. Pat. No. 4,029,095 issued Jun. 14, 1977). The Pena device was also heavy and inconvenient for the patient to administer the drug to treat diseases.

Babbitt et al. invented a portable device used to aspirate and remove fluids from nasal and sinus cavities (SINUS EVACUATOR APPARATUS, U.S. Pat. No. 4,403,611 issued Sep. 13, 1983). Since this device only applies a negative pressure to aspirate the sinus fluids from the nasal and sinus cavities, it cannot be used to deliver a drug into sinuses.

Liu, one of the current inventors, presented two inventions related to a nasal-nasopharyngeal-cleaning. (NNC) system used to remove harmful substances from a human's nasal and nasopharyngeal cavities (Liu, Nasal-nasopharyngeal-cleaning system, U.S. Pat. Nos. 6,238,377 May 29, 2001; and 6,736,792, May 18, 2004). However, the previous two inventions did not teach if the NNC system could deliver therapeutic agents into sinonasal cavities. Furthermore, collection of the out-flow liquid from sinonasal cavities was not taught. The current invention is to meet the special need of using a kit for delivering a functional liquid into sinonasal cavities, as well as nasal and nasopharynx, and to collect the drug or a vaccine in the out-flow liquid from sinonasal cavities.

Clinical diagnosis of respiratory tract infectious diseases relies on the laboratory test of the specimens collected from sinonasal cavities. Nasal wash is one of the most commonly used methods of collecting sinonasal specimen. The US Center for Diseases Control (CDC) (http://www.cdc.gov/flu/professionals/diagnosis/rapidlab.htm) and World Health Organization (WHO) http://www.who.int/csr/disease/avian_influenza/guidelines/humanspecimens/en/index.html provide guidelines for the collection of human specimens for laboratory diagnosis of influenza. The basic procedure is: a patient sits in a comfortable position with the head slightly tilted backward and is advised to keep the pharynx closed by saying “K” while the washing fluid (usually physiological saline) is applied to the nostril by a medical professional. With a transfer pipette, 1-1.5 ml of washing fluid is instilled into one nostril at a time. The patient then tilts the head forward and lets the washing fluid flow into a specimen cup or a Petri dish. The process is repeated with alternate nostrils until a total of 10-15 ml of washing fluid has been used. Dilute approximately 3 ml of washing fluid 1:2 in transport medium. This procedure would take as long as 30 minutes.

The US Department of Health and Human Services (DHHS) also provides guidelines (http://www.hhs.gov/pandemicflu/plan/sup2.html) for medical professionals to collect specimen with nasal wash. The procedure is to have a patient sits with head tilted slightly backward; instill 1 ml-1.5 ml of non-bacteriostatic saline (pH 7.0) into one nostril; flush a plastic catheter or tubing with 2 ml-3 ml of saline; Insert the tubing into the nostril parallel to the palate; aspirate nasopharyngeal secretions; repeat this procedure for the other nostril; and then collect the specimens in sterile vials.

The above standard procedures recommended by WHO, DHHS and CDC require a medical professional to perform the specimen collection and take a long time, and therefore, these procedures add healthcare cost significantly. The repeating process of instilling and aspirating a liquid requires a high level of cooperation from a patient. There is no means to secure the completeness and accuracy of nasal washing solution to flow to a collection container, which reduces the repeatability and reliability of the specimen collection and the result of the microorganism test. Furthermore, the patient will be under a stress during the specimen collection process. A new system to enable a patient to accurately and completely collect sinonasal specimen without stress will eliminate the above disadvantages. The disclosed invention provides a simple and reliable method to meet this need. By using the new device, a layman can do a better job than a medical professional in collecting nasal wash specimen of using the old system.

Medical professionals agree that administration of a therapeutic solution directly into sinuses will add treatment efficacy and reduce side effects for patients with sinus problems. This is especially beneficial to the patients with chronic rhinosinusitis. Currently, various types of nebulizer are used to administer therapeutic aerosols for the treatment of respiratory diseases (J S Lass et al. Expert Opinion on Drug Delivery September 2006, Vol. 3, No. 5, Pages 693-702; P W Barry, C O'Callaghan. Eur Respir J 1998; 12: 463-466). Although the use of aerosol therapy is commonly suggested in the treatment of paranasal disorders, it is difficult to achieve an effective delivery of drugs into sinuses. The investigators tried and shown that an oscillating airflow produced by phonation (nasal humming) causes a large increase in the gas exchange between the nose and the paranasal sinuses, hence, increased the delivery of an aerosolized drug into the paranasal sinuses (Maniscalco et al. Sounding airflow enhances aerosol delivery into the paranasal sinuses. European Journal of Clinical Investigation, Volume 36, Number 7, July 2006, pp. 509-513(5). According to the objective findings by sinus CT scan, even a modern nebulizer can barely deliver a therapeutic solution into sinuses. The new device of a continual-positive-pressure (CPP) delivery system as disclosed herein can greatly improve efficiency of delivering therapeutic agents to sinuses, provides advantages for administration of the therapeutic agents into sinuses. Hence, the current invention improves the efficacy of treatments for rhinosinusitis.

It is a great challenge for prevention and treatment of upper respiratory infections, such as the common cold and flu. For common cold viruses, antiviral drugs or vaccines are not available. The reason is that those drug candidates are less specific upon viral mutations and quickly become viral resistance. Chronic rhinosinusitis, another common disease, is lack of effective therapy due to complicated etiologies, multiple pathogens, antibiotic resistance, or due to adverse effects during a long-term use of antibiotics, antihistamines or anti-inflammatory drugs. Many herbs have been used for treating respiratory diseases for hundreds of years without noticeable side effects. For example, Mongolian dandelion, Fructus liquidambaris, Decumbent bugle, Cynanchun paniculatum and Rhodemyrtus tomentosa, have been administered orally after boiling the plant in water either singly or in combinations for treatment of respiratory diseases for years. A number of active components from the herbs have been extracted, isolated and identified using modern technologies. The new formulation, with a higher content of the active components, significantly improves the therapeutic effects of the original herbs after administered orally, intramuscularly or intravenously. The newly formulated therapeutic solution administered directly into sinuses by using the currently invented device revolutionizes the treatment of chronic rhinosinusitis.

### BRIEF DESCRIPTION OF THE DRAWINGS

These and other objects of the present invention will be apparent to those skilled in the art from the following description of the invention. The invention is described with reference to the following figures:

FIG. 1 is a drawing of the continual-positive-pressure (CPP) sinonasal liquid delivering and collecting device. It is a hand-squeezable device to deliver a therapeutic liquid into sinonasal cavities, and to collect the flow-out of the administered liquid drug to determine what is the exact amount of volume of the liquid drug staying in the sinonasal cavities. This device can also be used to collect wash-out specimen from sinonasal cavities. The squeezable bottle 1 varies in the liquid holding capacity. The liquid is transferred through a thin tube 2 to the one-directional liquid valve 4 and then to nostril fitting 6. The nostril fitting is connected to the cap 9 at the exchangeable point 8. When the user releases the bottle after forcing the liquid flow into nostril fitting, air will enter into the bottle through one-directional air valve 5 while no liquid can flow back into the bottle. This air valve 5 is to keep the bottle a positive pressure. There will be no negative pressure generated when the liquid is repeatedly forced to flow out. Valves 4 and 5 can be made as two parts or can be constructed as one part. A dusting cover 7 keeps the device clean. When a small volume of therapeutic solution is to be administered into sinonasal cavities, the solution can be held in a small test-tube like container 3, which can be attached onto the cap 9 through a fix-mean. The therapeutic solution in the container 3 will be forced to enter into the thin tube 2 and then flow into sinonasal cavities. The container 3 has a volume gradient and is a removable part. The container 3 is particularly useful for quantitative administration of an expensive pharmaceutical preparation, such as a monoclonal antibody, and/or a vaccine, into sinonasal cavities. Certain amount of administered liquid drug may flow out of sinonasal cavities. The flow-out liquid can be collected into the collecting container (right panel). The delivering panel and collecting panel is linked through an apparatus 13. The apparatus 13 is easily adjustable to make the distance between the two panels align with the user's two nostrils. The collecting panel has a nostril fitting 10. This nostril fitting has a larger open than that on the delivering panel. Depending on the volume of the specimen to be collected, the specimen can directly flow into the collecting bottle 12 or into the collecting tube 14 within the bottle. The collecting tube 14 is either a removable part in a bottle or a complete separated part. The collecting bottle has its cap 11 to support the nostril fitting and seal the collecting bottle. The collecting panel may be replaced with a funnel-cylinder like collector which is shown in FIG. 4.

FIG. 2 shows a detail of the liquid-air valve of the CPP sinonasal liquid delivery device. The nostril fitting 15 has a neck 16 and is connecting with the cap through a connecting mean. Part 17 is a hole to supply air. Part 18 is an air-liquid combinational valve which only allows liquid to flow upper wards but air only flow into the bottle. Part 19 is a support for the air-valve. Part 20 is a fixing mean to make parts 15, 16 and 19 to become a functional unit. Part 21 is a connecting mean between nostril fitting and the cap 22. Part 23 is the bottle. The test-tube like small container inside this bottle is the same as shown in FIG. 1.

FIG. 3 shows another design of the CCP sinonasal liquid delivery device. The basic device is similar to a regular spray bottle. The bottle 24 has an inner thin tube 25 (inside of the bottle and/or further inside of the test-tube like container, not show) for transferring solution from the bottle to the nostril fitting 27. The liquid is forced to flow from the bottle to nostril fitting when a user pulls and releases the trigger 26 repeatedly. The bottle connects the nostril fitting through the special cap 28. The test tube-like container to hold a small volume of a therapeutic solution attached to the cap shown in FIG. 1 (number 3) does not show here as it is inside of the bottle. The nostril fitting has all structural and functional features of the head of a spray bottle. In addition, it has an adjustable mean which enables the formation of the aerosol, fine misting or liquid through the nostril fitting; its hemisphere end can enable a tight seal between the device and a user's nasal opening. The body of the nostril fitting 27 can be straight or curved to ease the liquid administration into sinonasal cavities.

FIG. 4 shows a design of the sinonasal liquid collector. The collector has two parts, a wide-open funnel head 29 and a cylinder body 30. The wide-open part can be held beneath nostril to easily collect the out-flow liquid. The cylinder-like tube can be removed from the funnel head, and then capped for transferring the collected liquid or specimen to an analytic laboratory. The funnel head and the cylinder-tube can also be made as one piece. Depending on the purpose of the use, this sinonasal liquid collector can be sterilized or non-sterilized.

### DETAILED DESCRIPTION OF THE DRAWINGS

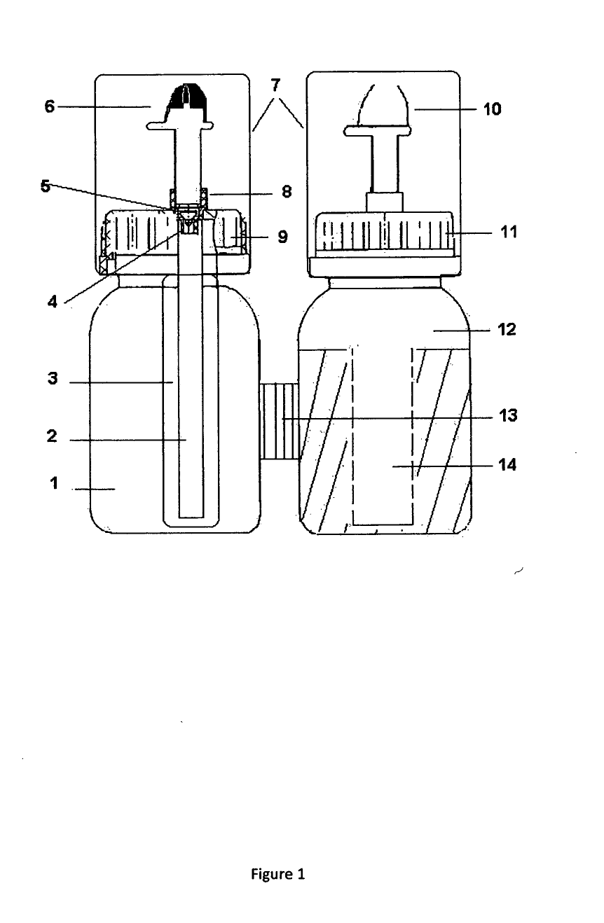

FIG. 1 is a drawing of the sinonasal liquid delivering and collecting device, which ensures a continual-positive pressure (CPP) in delivering a liquid drug into sinonasal cavities. It is a hand powered by the user, not necessarily by a medical professional. This device is also used to collect the flow-out of the administered liquid drug to determine the exact amount of volume of the liquid drug staying in the sinonasal cavities. This device can also be used to collect wash-out specimen from sinonasal cavities for determination of viruses, bacteria, fungi, inflammatory agents, eosinophils, pollen, etc. The squeezable bottle 1 varies in liquid holding capacity from 1 to 500 ml, preferably, from 50 to 400 ml, and more preferably, from 100 to 300 ml. The liquid is forced to flow through a thin tube 2, pass through the one-directional liquid valve 4, and then enter into nostril fitting 6, and finally, flow into sinonasal cavities. The nostril fitting is connected to the cap 9 at the exchangeable point 8. When the user releases the bottle after forcing the liquid flow into sinonasal cavities, air will enter into the bottle through a one-directional air valve 5 while no liquid can flow back into the bottle. This air valve 5 is to keep the bottle with a positive pressure. There will be no negative pressure generated when the liquid is repeatedly forced to flow out. Valves 4 and 5 can be made as two parts or can be constructed as one part. The valves can be placed in a varied position of the liquid route. Preferably, these valves are placed in or above the cap. When a small volume of therapeutic solution is to be administered into sinonasal cavities, the solution can be placed in a small test-tube like container 3, which can be attached onto the cap 9 through a fix-mean. The therapeutic solution in the container 3 will be forced to enter into the thin tube 2 and then flow into sinonasal cavities. The small test-tube like container 3 has a volume gradient and is a removable part. The small test-tube like container 3 is particularly useful for quantitative administration of an expensive pharmaceutical preparation and/or a vaccine into sinonasal cavities.

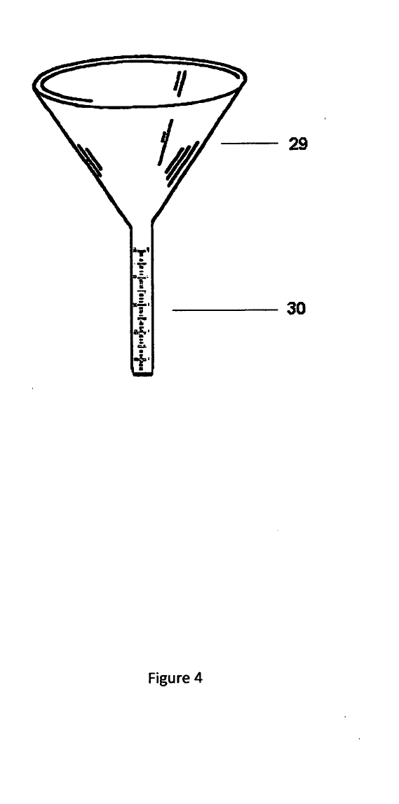

Certain amount of administered liquid drug may flow out of sinonasal cavities. The flow-out liquid can be collected into the collecting container (right panel). The delivering panel and collecting panel is linked through an apparatus 13. The apparatus 13 is easily adjustable to make the distance between the two panels aligning with the user's two nostrils. The collecting panel has a nostril fitting 10. This nostril fitting has a larger open than that on the delivering panel. Depending on the volume of the out-flow liquid to be collected, the out-flow liquid can directly flow into the collecting bottle 12 or into the collecting tube 14 within the bottle. The collecting tube 14 is either a removable part in a bottle or a complete separated part. The collecting bottle has its cap 11 to support the nostril fitting and seal the collecting bottle. The collecting panel may be replaced with a funnel-cylinder like collector which is shown in FIG. 4.

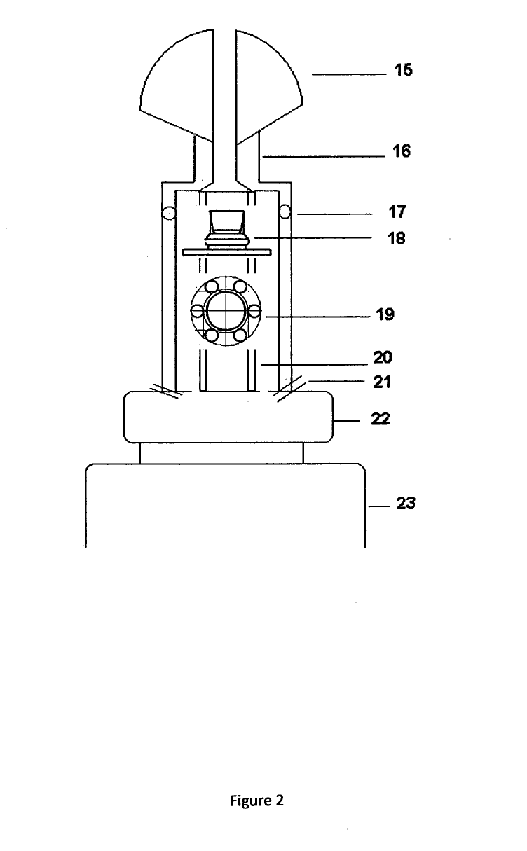

FIG. 2 shows a detail of the liquid-air valve of the CCP sinonasal liquid delivery device. The nostril fitting 15 has a neck 16 and is connecting with the cap through a connecting mean. After assembled, Parts 18 and 19 form a liquid-only route. Air cannot enter into this route. On this special nostril fitting, a number of holes 17 are on the hollow wall to supply air when the user is to release the squeezing power onto the bottle. Part 18 is an air-liquid combinational valve which only allows liquid to flow upper wards but air only flow into the bottle. Part 19 is a support for the air-valve. This part has multiple holes which allow air enter to the bottle when the flexible edge of the air valve 18 is to detach from it. Part 20 is a fixing mean to make parts 15, 16 and 19 to become a functional unit. Part 21 is a connecting mean between nostril fitting and the cap 22. When the nostril fitting is fixed to the cap, both the air chamber and liquid route are formed. Part 23 is the bottle. The test-tube like small container inside this bottle is similar to that shown in FIG. 1.

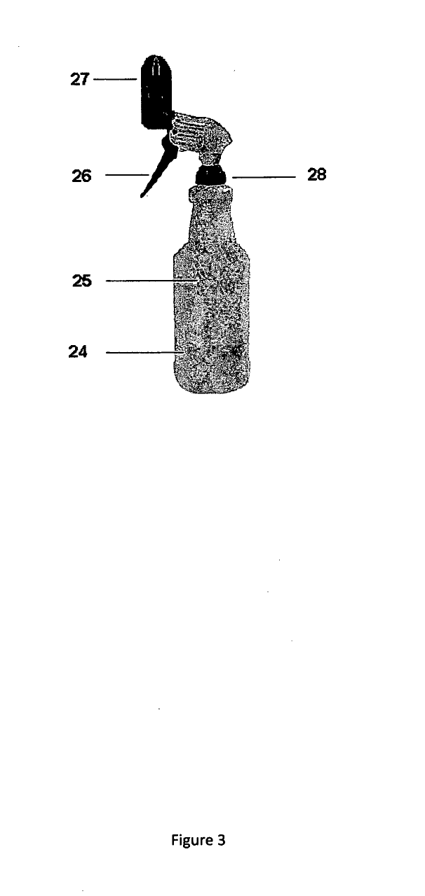

FIG. 3 provides another design of the delivering device. It can also ensure a continual-positive pressure (CPP) in delivering a liquid drug into sinonasal cavities. The basic device is similar to a regular spray bottle but the spray head becomes a multiple functional nostril fitting. The bottle 24 of the device has an inner thin tube 25 (inside of bottle, not show) to transfer liquid from the bottle to the nostril fitting 27. The liquid is forced to flow from the bottle to nostril fitting when the user activates the trigger 26. The test tube-like container to hold a small volume of a therapeutic solution attached to the cap similar to what is shown in FIG. 1 (number 3 which is not shown here as it is inside of the bottle). The bottle connects the nostril fitting through the multiple functional cap 28. The nostril fitting 27 has not only all structural and functional features of the head of a spray bottle, also has an adjustable mean which enables the formation of the aerosol, fine misting or liquid through the nostril fitting. The internal mechanical characteristics of the adjustable nostril fitting 27 can be created by a specially designed mean. Its hemisphere end of the nostril fitting 27 can enable a tight seal between the device and a user's nasal opening. The body of the nostril fitting 27 can be straight or curved to ease the liquid administration into sinonasal cavities. The length of the nostril fitting can vary from 0.5 cm to 20 cm, preferably from 2 to 15 cm, or more preferably, about 5 cm. The degree of the curve or straightness of the nostril fitting can vary in all directions and degrees to meet the need of a variety of users. This device enables the administration of a therapeutic liquid into a large surface of sinonasal cavities, while the traditional nasal sprayer could barely deliver the therapeutic agents to the sinuses. When this device is being used to collect sinonasal specimen, the collecting panel can be the same shown in FIG. 1, collecting panel, or may be a funnel-cylinder like collector shown in FIG. 4.

FIG. 4 shows a design of the sinonasal liquid collector. The collector has two parts, a wide-open funnel head and a cylinder body smoothly connected with a common mean. The wide-open part 29 can be held beneath nostril to easily and completely collect the out-flow liquid. The cylinder-like tube 30 can hold the out-flow liquid with a known volume. The cylinder-like tube can be removed from the funnel head after collecting out-flow liquid, then capped for transferring the collected liquid or specimen to an analytic laboratory. The shape and diameter of the funnel head can be varied reasonably to meet the user's need. The volume of the cylinder-tube can be varied from 1 to 500 ml; preferably, 10 to 200 ml. The funnel head and the cylinder-tube can also be made as one piece. Depending on the purpose of the use, this liquid collector can be sterilized or non-sterilized.

It is further disclosed that the CPP sinonasal liquid delivering and collecting device has the following features: 1) a delivery device is connected with a nostril fitting; 2) said the CPP delivery device is in the closed position nominally and opens to deliver a solution into sinonasal cavities upon activation; 3) said delivery device has a control means for quantitatively delivering a solution into sinonasal cavities; 4) said nostril fitting is a one-fit-all object to prevent leakage of the solution from nose openings; 5) said nostril fitting is part of an aerosol generator for delivering a therapeutic solution to the entire or part of the upper respiratory tract; 6) said nostril fitting is part of a liquid flow regulator to deliver a therapeutic solution into sinonasal cavities; 7) said nostril fitting is an exchangeable aerosol generator and a liquid flow regulator; 8) said CPP delivery bottle means has not only the function of a spray bottle also significantly adds at least three new features in the spray head, such as hemisphere end to become a nostril fitting, curved shape to ease the user in administering a therapeutic liquid in a tooth-brushing position; an inner mechanic mean to generate multiple forms of liquid, like flow liquid, aerosol or fine misting; 9) said CPP delivery device is powered or triggered by a human hand; 10) said CPP delivery device is to complete its function accompanied with a collecting panel; 11) said CPP delivery device disallows liquid back-flow; 12) said CPP delivery device can be made with all safe materials; 13) said CPP delivery device has a nostril fitting which is connected in any angle and in any appropriate length; 14) said CPP delivery device has a valve mean to ensure one-directional liquid flow; 15) said CPP delivery device has a valve mean to maintain a positive pressure within the delivery bottle; 16) said CPP delivery device can be connected with a tooth brushing mean to be used for cleaning both sinonasal and oral cavities; 17) said CPP delivery device is a pump-like apparatus; 18) said CPP delivery bottle means connected with another set of a nostril fitting connecting with a container for collecting the outflow liquid; 19) said CPP delivery device is connected with another container for collecting nasal wash specimen by a user; 20) said CPP delivery device is a system to be used to administer a functional liquid to sinonasal cavities; 21) said CPP delivery device is a system to be used to administer a functional liquid to sinonasal cavities in reducing the load of pathogenic microbes without causing drug resistance; 22) said CPP delivery device is a system to be used to administer a functional liquid to sinonasal cavities in reducing the content of inflammatory agents without disturbing the body's immune function; 23) said CPP delivery device is a system to be used to administer a functional liquid to sinonasal cavities in treating respiratory diseases without causing adverse events; 24) said CPP delivery device is a system to be used to administer a functional liquid to sinonasal cavities in treating other human diseases without causing the time of a medical professional.

### BRIEF SUMMARY OF THE INVENTION OF THE COMPOSITION AND METHOD OF USE

These and other objects of the present invention will be apparent to those skilled in the art from the following description of the invention.

The present invention provides a pharmaceutical composition comprising an extract or combination of extracts having an antiviral, antibacterial, antifungal, anti-inflammatory, anti-allergy or immunomodulating property, wherein the extract or the combination of extracts is obtained from a single or a combination of plants, wherein the plant includes Mongolian dandelion, Fructus liquidambaris, Decumbent bugle, Cynanchun paniculatum, and Rhodemyrtus tomentosa (DLBPT). This extract is referred to herein specifically as the DLBPT extract.

The present invention further provides a pharmaceutical composition having an antiviral, antibacterial, antifungal, anti-inflammatory, anti-allergy or immunomodulating property, comprising at least one of taraxocerin, liquidambaric acid, ajugasterones, paeonolum, and gelomyrtol (TLAPG), particularly in isolated and purified form. This formulation is referred to herein specifically as the TLAPG formula.

The present invention further provides a compound of taraxocerin, liquidambaric acid, ajugasterones, paeonolum and gelomyrtol, optionally having a substituent selected from the group consisting of HCOH or COOH and an amide, ester, and salt thereof, cyano, amino, substituted amino, alkyl, hydroxyalkyl, hydroxy, alkyloxy, alkyl carbonyl, alkyl carbonyloxy, alkyloxy carbonyl, halo, sulfate, and nitro; wherein one or more aromatic hydrogens may be replaced with a substituent selected from the group consisting of hydroxy, cyano, amino, substituted amino, alkyl, hydroxyalkyl, halo, sulfate and nitro.

The present invention further provides a pharmaceutical composition comprising a pharmaceutically acceptable carrier and a compound of taraxocerin, liquidambaric acid, ajugasterones, paeonolum and gelomyrtol.

The present invention further provides a method of treating or preventing respiratory disorders, such as common cold, influenza, acute or chronic airway infection, airway inflammation, airway allergy, asthma, or chronic rhinosinusitis, or treating or preventing cardiovascular diseases, of a mammal through administering to sinonasal cavities of a mammal a pharmaceutical composition of the present invention.

The present invention further provides a process for preparing the DLBPT extract comprising the steps of:
(a) extracting a single plant or mixture of Mongolian dandelion, Fructus liquidambaris, Decumbent bugle, Cynanchun paniculatum, and Rhodemyrtus tomentosa with water at a temperature above 20 degree C. to obtain a water or organic extract;
(b) concentrating the extract to obtain a concentrate;
(c) purifying the concentrate on a chromatographic column;
(d) recovering the purified fractions;
(e) lyophilizing the fractions to a dry powder form.

The present invention further provides a delivery system for administrating DLBPT extract or TLAPG formula to the sinonasal cavity. The delivering method comprises a CPP liquid delivery mean connecting with a nostril fitting to deliver aerosol or liquid into sinonasal cavities of a mammal a pharmaceutical composition of the present invention.

While the invention has been described and disclosed below in connection with certain preferred embodiments and procedures, it is not intended to limit the invention to those specific embodiments. Rather it is intended to cover all such alternative embodiments and modifications as fall within the spirit and scope of the invention.

Thus, here is to further disclose a delivering device to administer a liquid into sinonasal cavities. The invention comprises a therapeutic solution, a CPP delivery system and a nostril fitting, and a nasal out-flow collector. The said adjustable nostril fitting can be used to deliver aerosol, fine misting, or liquid when the user to loose or tighten the end part of the adjustable nostril fitting.

### DETAILED DESCRIPTION OF THE INVENTION OF THE COMPOSITION AND METHOD OF USE

The present invention provides a pharmaceutical composition comprising an extract or combination of extracts having an antiviral, antibacterial, antifungal, anti-inflammatory, anti-allergy or immunomodulating property. The extract or combination of extracts is obtained from a single plant or the combination of plants wherein the plant includes at least one of Mongolian dandelion, Fructus liquidambaris, Decumbent bugle and Rhodemyrtus tomentosa. Any suitable species of these plants can be used to obtain the extract or combination of extracts for the inventive pharmaceutical composition. Any suitable part of the plant can be used. For example, leaves, twigs, branches, bark, roots, flowers, and fruits can be used.

The present invention further provides a process for preparing a DLBPT extract and TLAPG formula. The DLBPT extract can be prepared by any suitable method. For example, an extract can be prepared from each plant and the combined plants. The DLBPT extract can be prepared by suitably combining the extracts from the other plants.

The DLBPT extract can have any suitable combination of the plant parts. For example, the plant parts can be in the ratio of 1:1:1:1:1 or 1:1:2:2:4, respectively, or in any suitable ratio, among Mongolian dandelion, Fructus liquidambaris, Decumbent bugle, Cynanchun paniculatum, and Rhodemyrtus tomentosa extracts.

The present invention further provides a pharmaceutical composition comprising taraxocerin, liquidambaric acid, ajugasterones, paeonolum and gelomyrtol, particularly in isolated and purified form. Taraxocerin, liquidambaric acid, ajugasterones, paeonolum and gelomyrtol, singly and in combination, is effective as a component of the pharmaceutical composition of the present invention.

The concentrations of the active ingredients can be standardized for the DLBPT extract. The therapeutic effect therefore is repeatable as the active components are within the allowable variation.

As compared to the original plants, smaller concentrations of the active ingredients can be used in TLAPG formula. Any possible side effect is minimized because of the greater purity of the ingredients and the reduced concentration of the other non-active components.

The present invention further provides a process for isolation of taraxocerin, liquidambaric acid, ajugasterones, paeonolum and gelomyrtol in a purified form from a plant material containing the same. Any suitable water and organic solvent, preferably distilled water and ethanol, can be used in the extraction and purification process. Below are those basic steps:
(a) extracting a single plant or mixture of Mongolian dandelion, Fructus liquidambaris, Decumbent bugle, Cynanchun paniculatum, and Rhodemyrtus tomentosa with a water and then an organic solvent at a temperature above 20 degree C. to obtain an extract;
(b) concentrating the extract to obtain a concentrate;
(c) purifying the concentrate on a chromatographic column, or physical and chemical means;
(d) recovering the'purified fractions;
(e) removing solvent to obtain a purified component.

The pharmaceutical compositions of the present invention are particularly suitable for treatment or prevention of infections by viruses that affect the respiratory system. Particular examples of these viruses include rhinoviruses, influenza viruses A, B and C, parainfluenza virus types 1, 2, 3, and 4, respiratory syncytial viruses, Epstein-Barr virus. Hepatitis viruses are also inhibited by the present invention.

The pharmaceutical compositions of the present invention are particularly suitable for the treatment or prevention of infections by bacteria selected from the group consisting of Streptococcus pneumoniae, Heamophilus influenzae, Vibrio metchnikovii, Edwardsiella tarda, Proteous vulgaris, Shigalla flexneri, Shigalla sonnei, Staphylococcus aureus, Proteus mirabilis, Citrobacter freundil, Group D streptococcus, P. shigelloides, Aeromonas hydrophilia, Salmonella typhi, Pseudomonas aeruginosa, E. coli, H. alvei, Enterobacter cloacea, Staphylococcus epidermis, Branhamella, and Bacillus subtilis.

In addition, the pharmaceutical compositions of the present invention are also suitable for the treatment or prevention of the infection caused by fungus. The pharmaceutical compositions of the present invention are also suitable for the treatment or prevention of a mixed infection by virus, bacterium, and a fungus.

The pharmaceutical compositions of the present invention are further capable of eliminating inflammatory agents in the respiratory tract, enhancing the natural mucociliary clearance, promoting mucolytic activity, reducing airway resistance, easing inspiration and expiration, and increasing blood oxygenation of the lung.

The present invention further provides a method of treatment or prevention of respiratory disorders in a mammal comprising administering to said mammal a pharmaceutical composition of the DLBPT extract or TLAPG formula.

The present invention further provides a method of treating or preventing the other disorders, such as cardiovascular diseases or hypertension, in a mammal comprising administering to said mammal a pharmaceutical composition of the DLBPT extract or TLAPG formula.

The present invention further provides a pharmaceutical composition comprising a pharmaceutically acceptable carrier and an effective (e.g., therapeutically or prophylactically effective) amount of at least one of the compounds set forth above.

The present invention further provides a method of treatment and/or prevention of respiratory disorders in a mammal by administering to the mammal an effective amount (e.g., therapeutically or prophylactically effective) of at least one of the compounds set forth above.

The present invention further provides a method of treatment and/or prevention of respiratory disorders in a mammal by administering to the mammal an effective amount (e.g., therapeutically or prophylactically effective) of at least one of the compound set forth above, in addition to an aloe extract.

The present invention further provides a method of treatment and/or prevention of respiratory disorders in a mammal by administering to the mammal an effective amount (e.g., therapeutically or prophylactically effective) of at least one of the compound set forth above, in addition to the extract of peppermint (Behe).

The present invention further provides a method of treatment and/or prevention of respiratory disorders in a mammal by administering to the mammal an effective amount (e.g., therapeutically or prophylactically effective) of at least one of the compound set forth above, and the extract of Lucid ganoderma (Lingzhi).

The present invention further provides a method of treatment and/or prevention of respiratory disorders in a mammal by administering to the mammal an effective amount (e.g., therapeutically or prophylactically effective) of at least one of the compound set forth above, and the extract of Honeysuckle flower (Jinyinghua).

The present invention further provides a method of treatment and/or prevention of respiratory disorders in a mammal by administering to the mammal an effective amount (e.g., therapeutically or prophylactically effective) of at least one of the compound set forth above, and the extract of Weeping forsythia (Lianqiao).

The present invention further provides a method of treatment and/or prevention of respiratory disorders in a mammal comprising administering to the mammal an effective amount (e.g., therapeutically or prophylactically effective) of at least one of the compound set forth above, and the extract of Fimbriate orostachys (Wuasong).

The present invention further provides a method of treatment and/or prevention of respiratory disorders in a mammal by administering to the mammal an effective amount (e.g., therapeutically or prophylactically effective) of at least one of the compound set forth above, and the extract of Prunella vulgaris (Xiakucao).

The present invention further provides a method of treatment and/or prevention of respiratory disorders in a mammal by administering to the mammal an effective amount (e.g., therapeutically or prophylactically effective) of at least one of the compound set forth above, and the extract of Scutellariae baicalensis (Huangqin).

The present invention further provides a method of treatment and/or prevention of respiratory disorders in a mammal by administering to the mammal an effective amount (e.g., therapeutically or prophylactically effective) of at least one of the compound set forth above, and the extract of Chrysanthemum indicum (Yejuhua).

The present invention further provides a method of treatment and/or prevention of respiratory disorders in a mammal by administering to the mammal an effective amount (e.g., therapeutically or prophylactically effective) of at least one of the compound set forth above, and the extract of Mulberry leaves (Shangye).

The pharmaceutically acceptable carriers described herein, for example, vehicles, adjuvants, excipients, or diluents, are well-known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there are a wide variety of suitable formulations of the pharmaceutical composition of the present invention. The following formulations for oral, intranasal, intrasinal, intrathecal, aerosol, parenteral, subcutaneous, intravenous, intraarterial, intramuscular, interperitoneal, rectal, and vaginal administration are merely exemplary and are in no way limiting.

Formulations suitable for oral administration can consist of (a) solutions, such as an effective amount of the compound dissolved in diluents, such as water, saline, milk or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compound of the present invention, alone or in combination with other suitable components, can be made into aerosol formulations to be administered via inhalation. These aerosol formulations can be placed into pressurized acceptable propellants, such as dichlorodifluoromethane. They may also be formulated as pharmaceuticals for non-pressured preparations, such as in a nebulizer or an atomizer. It is observed that the present invention can deliver the drug to a broader surface area of sinonasal cavities than using a nebulizer or an atomizer. Therefore, the active components from the plants may be formulated as pharmaceuticals and administered by using the present invention for treating rhinosinusitis, and other sinonasal disorders.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

The parenteral formulations will typically contain from about 0.1 to about 25% by weight of the active ingredient in solution. Suitable preservatives and buffers can be used in such formulations. In order to minimize or eliminate irritation at the site of injection, such compositions may contain one or more nonionic surfactants. The added surfactants will also increase the therapeutic efficacy for treating respiratory tract disorders by using the present invention. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art, Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4th ed., pages 622-630 (1986).

Additionally, the compounds of the present invention may be made into suppositories by mixing with a variety of bases, such as emulsifying bases or water-soluble bases. Formulations suitable for vaginal administration may be presented as tampons, creams, gels, pastes, foams, or spray formulas containing, in addition to the active ingredient, such carriers as are known in the art to be appropriate.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. For convenience, the total daily dosage may be divided and administered in portions during the day if desired. In proper doses and with suitable administration of certain compounds, the present invention provides for a wide range of responses. The dosages range from about 0.001 to about 1000 mg/kg body weight of the mammal being treated per day. Preferred dosages range from about 0.01 to about 10 mg/kg body weight/day, and further preferred dosages range from about 0.01 to about 1 mg/kg body weight/day.

The present invention provides a delivery system to administer a liquid formulation directly into sinonasal cavities. Pharmaceutical preparations, such as a vaccine or a therapeutic agent, can be administered into sinonasal cavities, especially into sinuses by using this newly invented device. This is a significant advantage as compared to a nebulizer by which the drug is hard to be delivered into sinuses. The procedures are: remove the cap from the bottle; add the liquid drug to the bottle, or add the powder drug and dilution solution into the bottle; tighten cap; place the nostril fitting of delivering the liquid snugly into one nostril; open mouth and breathe gently through the mouth; bending forward; squeeze the bottle gently to make the liquid drug flow slowly into nasal and sinus cavities. If the liquid drug has a small volume, one can do the following: remove the cap from the bottle; insert the transferring tube into the container holding a drug in liquid form; place the container straight-up within the bottle; tighten cap; place the nostril fitting snugly into one nostril; open mouth and breathe gently through the mouth; bending forward; squeeze the bottle gently to make the liquid drug flow slowly into nasal and sinus cavities.

Delivering the drug to the lung using the present invention is to perform the following procedures: remove the cap from the bottle; add the liquid drug to the bottle; tighten cap; place the nostril fitting of delivering the aerosol or fine misting snugly into one nostril; open mouth and breathe gently through the mouth; bend forward; squeeze the bottle gently to make the aerosol and fine misting of the drug be inhaled into the lung.

The CPP liquid delivery system can also be used to reduce the load of disease-causing agents, such as infectious microorganisms, pollens, and other allergic agents as well as toxic chemicals in the sinonasal cavities. It can also be used to prevent these infectious agents from presenting in aerosols/droplets. The invented system may be applied by an individual to himself or herself as often as needed, or as required, or deemed convenient, or used by a medical professional to clean sinonasal cavities before and after a surgery procedure. The method of use is: add the washing solution to the bottle, or dissolve the powder mix with warm water; shake well; place the nostril fitting snugly into one nostril; open mouth and breathe gently through mouth; bend forward over a sink so that the tip of the nose is the lowest point of the head; squeeze the bottle gently to make the liquid flow slowly until liquid runs from the other nasal passage. Then remove nostril-fitting and then insert to the other nostril. Repeat the above steps so the washing liquid will flow out from the first-time inserted nostril. The nasal cavities, sinuses, nasopharyngeal cavities and oropharyngeal cavities will be cleaned systemically.

The disclosed system can also be used to collect sinonasal specimen from a mammal. The invented system can be applied by an individual to himself or herself as often as needed, or as required, or deemed convenient. It can also be used by a medical professional to obtain a sinonasal specimen for diagnosis and treatment of a patient. The using method is: add the liquid to the bottle; place the nostril fitting of in-flow snugly into one nostril and the nostril-fitting of out-flow into another nostril; open mouth and breathe gently through mouth; bend forward; squeeze the bottle gently to make the liquid flow slowly until liquid runs from the other nasal passage; the other nostril fitting with a large open is connected to a collecting container. The wash solution is then processed by a medical or lab professional. This procedure only takes about 2 to 5 minutes for running through up to 200 ml of liquid. All nasal wash liquid is completely collected without spill. It saves the time for both a medical professional and a patient, in addition to the completeness and a high level of repeatability of nasal wash. The advantage of sinonasal specimen collection by using the present invention is obvious as compared to the procedure currently recommended by the WHO, DHHS or CDC, which costs a medical professional a considerable time without a higher reproducibility.

The following examples further illustrate the present invention but, of course, should not be construed as in any way limiting its scope.

### Example 1

This example illustrates the efficacy of the DLBPT extract which was formulated and administered intranasally for treating chronic rhinosinusitis. The DLBPT extract was prepared by combining the extract of Mongolian dandelion and aloe. A patient with 30 years of history of chronic rhinosinusitis conducted a self-observation. Before intranasal application of the solution, he had a chronic nasal obstruction and short of breath. Three times after he administered the DLBPT extract using the CPP delivery device, he reported that his symptoms of the nasal obstruction were improved, and he could breathe much easier than before. After five times of use, he reported that his brain thinking power became much better than before since he slept well after nasal obstruction was disappeared. He then used the intranasal solution on a daily basis for three months without side effects.

### Example 2

This example illustrates the efficacy of the DLBPT extract which was formulated and administered intranasally using the CPP delivery device for treating chronic sinus obstruction. The DLBPT extract was prepared by combining the extract of Fructus liquidambaris and aloe. A patient with many years of history of chronic sinusitis planned to have a nasal surgery to treat sinus obstruction after a sinus CT-scan confirmed chronic rhinosinusitis. Before scheduled the surgery, he performed a self-comparison of nasal irrigation by using the CPP system to deliver therapeutic solution. Two times after administration of the DLBPT extract, he felt that the symptoms of the nasal obstruction and headache were significantly improved. After continuing use of the preparation for two months, his quality of life was significantly improved, and he did not experience any side effects. There is no need for him to be treated surgically.

### Example 3

This example illustrates the efficacy of the DLBPT extract which was formulated and administered intranasally using the CPP delivery device for treating common cold. The DLBPT extract was prepared by combining the extract of Mongolian dandelion, Rhodemyrtus tomentosa and aloe. Two days after participating a social gathering, an adult man developed common cold symptoms. He administered the intranasal solution twice a day for three days. His common cold symptoms were suppressed to a minimum and no more symptoms on day 4 after the common cold onset.

### Example 4

This example illustrates the administration of a therapeutic solution into sinuses. The DLBPT extract was formulated and administered intranasally using the CPP delivery device before a CT scan. The CT films confirmed that after intranasal administration of the therapeutic solution using the present. CPP intranasal delivery device, the liquid present in all 4 pairs of paranasal sinuses. The maxillary and ethmoid sinuses have more volume of the administered liquid drug, while the sphenoid and frontal sinuses have less liquid drug based on the CT scan result.

### Example 5

This example illustrates the procedure of using the present invention to collect sinonasal specimen from a man. The invented system was used by a non-medical professional male adult. He opened the sinonasal specimen collection kit and read the instruction. He then added the 100 ml of sterilized normal saline to the bottle; placed the in-flow nostril fitting into one nostril and the out-flow nostril-fitting into the other nostril. He opened his mouth and breathed gently through mouth; bended forward slightly. After squeezing the bottle for one second, the liquid flew through sinonasal cavities and entered into the collecting container. He completed the 50 ml nasal wash specimen collection procedure within 5 minutes. The other un-used 50 ml saline was discarded.

### Example 6

This example illustrates the efficacy of using the present invention to remove inflammatory mediators from sinonasal cavities of 6 patients with chronic rhinosinusitis and 5 healthy adults. The patients and healthy adults administered the 240 mL therapeutic irrigation liquid to their sinonasal cavities and collected the first 50 mL of the nasal wash specimens. Those specimens were then processed by a clinical lab for observing the presence of eosinophils and degradation debris. The nasal wash specimens from the 6 patients with chronic rhinosinusitis had positive eosinophils. On the other hand, the inflammatory cells were not detected in the nasal wash specimens from the healthy adults. The entire procedure did not cause any adverse events.

### Example 7

This example illustrates the efficacy of using the present invention to remove fungi from sinonasal cavities of 6 patients with chronic rhinosinusitis and 5 healthy adults same as above in Example 6. The nasal wash specimens were also processed by a clinical lab for detecting the presence of fungi in sinonasal specimen. The nasal wash specimens from the 6 patients with chronic rhinosinusitis, as well as the 3 out of 5 normal healthy adults, had Alternaria alternata. The amount of detectable fungi become less and less after subsequent procedures were performed. This indicated that practice of sinonasal irrigation with the CPP intranasal drug delivery device and the therapeutic solution reduced the detectable fungi in the sinonasal cavities. The 6 patients also reported the significant improvement of their symptoms. The entire procedures for all trial subjects did not cause any adverse events.

### Example 8

This example illustrates the efficacy of using the present invention to remove bacteria from sinonasal cavities of 6 patients with chronic rhinosinusitis and 5 healthy adults same as above in Example 6. The nasal-wash specimens were also processed by a clinical lab for detecting the presence of bacteria. The nasal wash specimens from the 6 patients with chronic rhinosinusitis, as well as the 4 out of the 5 normal healthy adults, contained the detectable Streptococcus pneumoniae. The amount of detectable bacteria become less and less after subsequent procedures were performed. The entire procedure did not cause any antibiotic resistance, or other adverse events.

### INDUSTRIAL APPLICABILITY

Through the easy use of the CPP delivery device to administer a pharmaceutical composition of this invention, the general population now has a simple, safe and inexpensive method that can be used to administer a therapeutic solution into sinonasal cavities to treat and prevent a human disease. This CPP device can also be used to remove inflammatory agents from the sinonasal cavities by a layman. For a medical professional, she/he can show to the patient how to use this simple and accurate method to collect nasal wash specimen. As mentioned previously, routinely use of this CPP device will reduce pathogenic microorganism loads and thereby reduce the spread of infection to others, and reduce the opportunity for the disease causing agents to reach his or her own lower respiratory tract of the carrier. The significant advantage of using this new system is that there will be no chance to develop multiple-drug resistance, which is a big public threat in light of many deaths caused by those super bugs. This system also helps the patient to remove harmful inflammation mediators from sinonasal cavities without using those anti-inflammatory drugs. Hence, those drug's adverse effects and disturbance of immunity can be avoided. The medical community and the general population will greatly benefit from the composition, device and method disclosed herein. The medical community now has a simple and reliable method to let the patient to administer a variety of therapeutic agents into sinonasal cavities to treat those disorders.

Those skilled in the art will appreciate that changes and modifications can be made to the device, the preferred embodiment of pharmaceutical composition, and the methods of use disclosed herein without departing from the spirit and scope of the present invention as set forth as defined by the following claims.

### REFERENCES

| | | | |
|---|---|---|---|
| 1,248,891 | December 1917 | Nichols | 604/289. |
| 1,487,252 | March 1924 | Lore | 604/289. |
| 1,502,163 | July 1924 | Sprague | 604/217. |
| 2,566,806 | September 1951 | Miller | 128/173. |
| 2,582,529 | January 1952 | Curry | 128/200. |
| 2,693,800 | November 1954 | Caldwell | 604/289. |
| 3,847,145 | November 1974 | Grossan | 601/160. |
| 4,029,095 | June 1977 | Pena | 128/250. |
| 4,403,611 | September 1983 | Babbitt et al. | 604/118. |
| 4,980,163 | December 1990 | Blackburn et al. | 434/94. |
| 5,116,311 | May 1992 | Lofstedt | 604/54. |
| 5,183,467 | February 1993 | Mouney | 604/140. |
| 5,314,690 | May 1994 | Patterson et al. | 424/114. |
| 5,505,193 | April 1996 | Ballini et al. | 128/200. |
| 5,752,510 | May 1998 | Goldstein | 128/200. |
| 5,794,619 | August 1998 | Edelman et al. | 128/200. |
| 6,238,377 | May 2001 | Liu | 604/289. |
| 6,736,792 | May 2004 | Liu | 598/522. |

### Other References

A publication entitled: “Ephedrine-saline nasal wash in allergic rhinitis”, in J. Allergy Clin. Immunol., by Wiqar A. Shaikh, M.D., November, 1995 p. 597-600.
A publication entitled: “Parainfluenza viral infections in children: Correlation of shedding with clinical manifestations” by Hall et al. in The Journal of Pediatrics, 1982, Vol. 91, No. 2, pp. 194-198.
A publication entitled “Clinically Useful Method for the Isolation of Respiratory Syncytial Virus” by Hall et al. in The Journal of Infectious Diseases, 1975, Vol. 131, No. 1, pp. 1-5.
A publication entitled: “Quantitative Shedding Patterns of Respiratory Syncytial Virus in Infants” by Hall et al. in The Journal of Infectious Diseases, 1975, Vol. 132, No. 2, Aug., pp. 151-156.
A publication entitled: New advances in aerosolised drug delivery: vibrating membrane nebuliser technology by Lass et al. in Expert Opinion on Drug Delivery 2006, Vol. 3, No. 5, Pages 693-702.
A publication entitled: A Novel Method of Drug Delivery for Fibrinolysis with Ho:YAG Laser-Induced Liquid Jet by Hirano et al. in Lasers in Medical Science 2002, 17(3).
A publication entitled: Sounding airflow enhances aerosol delivery into the paranasal sinuses by Maniscalco et al. in European Journal of Clinical Investigation, 2006, Vol. 36, Number 7, July 2006, pp. 509-513(5).
A publication entitled: Drug output from nebulizers is dependent on the method of measurement. P W Barry, C O'Callaghan. Eur Respir J 1998; Vol. 12: 463-466.
A publication entitled: Sounding airflow enhances aerosol delivery into the paranasal sinuses. Maniscalco et al. European Journal of Clinical Investigation 2006, Vol. 36, Number 7, pp. 509-513(5).
A publication entitled: Pharmaceutics and Pharmacy Practice, J.B. Lippincott Co., Philadelphia, Pa., Banker and Chalmers, eds., pages 1982, 238-250.
A publication entitled: Expert Opinion on Drug Delivery, J S Lass et al. September 2006, Vol. 3, No. 5, Pages 693-702.
A publication entitled: ASHP Handbook on Injectable Drugs, Toissel, 4th ed., 1986, pages 622-630.
A publication entitled: The American Academy of Otolaryngology-Head and Neck Surgery (AAO-HNS), http://www.entnet.org/healthinfo/sinus/sinus_questions.cfm, 2007.
A publication entitled: The diagnosis and incidence of allergic fungal sinusitis, Ponikau, J. U., D. A. Sherris, E. B. Kern, H. A. Homburger, E. Frigas, T. A. Gaffey, and G. D. Roberts. 1999. Mayo Clin. Proc. 74:877-884.
A publication entitled: Pharmacy Practice, J.B. Lippincott Co., Philadelphia, Pa., Banker and Chalmers, eds., 1982, pages 238-250.
A publication entitled: ASHP Handbook on Injectable Drugs, 1986, Toissel, 4th ed., pages 622-630.
A publication entitled: Microbiology of the maxillary and ethmoid sinuses in patients with chronic rhinosinusitis submitted to functional endoscopic sinus surgery. Nigro et al. Rev Bras Otorrinolaringol 2006, Vol. 72(2):217-222.

What is claimed is:

1. A pharmaceutical composition comprising:
a pharmaceutically acceptable carrier;
at least one of the compounds comprising taraxocerin, liquidambaric acid, ajugasterones, paeonolum and gelomyrtol;
at least one component of aloe, peppermint, Lucid ganoderma, Honeysuckle flower, Weeping forsythia, Small centipede, Membranous milkvetch root, Largehead Atractylodes rhizome, Prunella vulgaris, Scutellariae baicalensis, Chrysanthemum indicum, Mulberry leaves, and Fimbriate orostachys.

2. The pharmaceutical composition of claim 1, wherein the said at least one component is extracted individually or in combination from Mongolian dandelion, Fructus liquidambaris, Decumbent bugle, Paniculate swallowwort root or Rhodemyrtus tomentosa.

3. The pharmaceutical composition of claim 1, wherein the said at least one component is chemically synthesized from any precursors of taraxocerin, liquidambaric acid, ajugasterones, paeonolum, or gelomyrtol.

4. The pharmaceutical composition of claim 1, wherein the said at least one of the compounds are chemically modified in hydroxy, alkyloxy, alkyl carbonyl, alkyl carbonyloxy, alkyloxy carbonyl, halo, cyano, nitro, sulfate or amino group.

5. A continual positive pressure sinonasal delivery system comprising:
a container for holding a pharmaceutical preparation in the liquid form;
a nostril fitting for delivering at least one of the formulations of liquid, aerosol, or mist;
a cap for seating the said nostril fitting on the top and for attaching the said squeezable container to the inner surface;
one pair of the air and liquid valves in the correct opposite flow directions to maintain continual positive pressure within the said squeezable container and to prevent backflow liquid contamination;
or the continual positive pressure is provided by a hand-triggered sprayer;
and a tube for transferring liquid from the said container to the liquid valve;

6. The continual positive pressure sinonasal delivery system in accordance with claim 5, wherein the nostril fitting is used for delivering a pharmaceutical composition into sinonasal cavities of a mammal.

7. The continual positive pressure sinonasal delivery system in accordance with claim 5, wherein the nostril fitting is used for delivering a biological preparation into sinonasal cavities of a mammal.

8. The continual positive pressure sinonasal delivery system in accordance with claim 5, wherein the nostril fitting is used for delivering a pharmaceutical composition comprising a pharmaceutically acceptable carrier and at least one of the compounds comprising taraxocerin, liquidambaric acid, ajugasterones, paeonolum and gelomyrtol to a mammal.

9. The continual positive pressure sinonasal delivery system in accordance with claim 5, wherein the nostril fitting is used for delivering into the sinonasal cavities of a mammal a pharmaceutical composition comprising a pharmaceutically acceptable carrier, and at least one of the individual components extracted from Mongolian dandelion, Fructus liquidambaris, Decumbent bugle, Paniculate swallowwort root, Rhodemyrtus tomentosa, aloe, peppermint, Lucid ganoderma, Honeysuckle flower, Weeping forsythia, Small centipede, Membranous milkvetch root, Largehead Atractylodes rhizome, Prunella vulgaris, Scutellariae baicalensis, Chrysanthemum indicum, Mulberry leaves, or Fimbriate orostachys.

10. A method of using a continual positive pressure sinonasal delivery system comprising:
a squeezable container for holding a pharmaceutical preparation in the liquid form;
a nostril fitting for delivering at least one of the formulations of liquid, aerosol, or mist;
a cap for seating the said nostril fitting on the top and for attaching the said squeezable container to the inner surface;
one pair of the air and liquid valves in the correct opposite flow directions to maintain continual positive pressure within the said squeezable container and to prevent backflow liquid contamination;
or the continual positive pressure is provided by a hand-triggered sprayer;
and a tube for transferring liquid from the said squeezable container to the liquid valve;
to deliver a pharmaceutical composition into the sinonasal cavities of a mammal to treat or prevent a disorder of a mammal.

11. The continual positive pressure sinonasal delivery system in accordance with claim 10, wherein the said disorder of a mammal includes but not limits to an infectious disease.

12. The continual positive pressure sinonasal delivery system in accordance with claim 10, wherein the said disorder of a mammal includes but not limits to an inflammatory disease.

13. The continual positive pressure sinonasal delivery system in accordance with claim 10, wherein the said disorder of a mammal includes but not limits to an immunological disorder.

14. The continual positive pressure sinonasal delivery system in accordance with claim 10, wherein the said disorder of a mammal includes but not limits to a metabolic disorder.

15. The continual positive pressure sinonasal delivery system in accordance with claim 10, wherein the said disorder of a mammal includes but not limits to a malignant growth disorder.

16. A method for treating rhinosinusitis comprising the steps of:
using a continual positive pressure sinonasal delivery system delivering to the sinonasal cavities of a mammal a therapeutic solution containing at least one of the components extracted from Mongolian dandelion, Fructus liquidambaris, Decumbent bugle, Paniculate swallowwort root, Rhodemyrtus tomentosa, aloe, peppermint, Lucid ganoderma, Honeysuckle flower, Weeping forsythia, Small centipede, Membranous milkvetch root, Largehead Atractylodes rhizome, Prunella vulgaris, Scutellariae baicalensis, Chrysanthemum indicum, Mulberry leaves, or Fimbriate orostachys;
wherein the sinonasal delivery system comprises a continual positive pressure and anti-backflow mechanism for reducing simultaneously the presence of the pathogenic microbe and the inflammatory mediator in sinonasal cavities of a mammal.

17. The method for treating rhinosinusitis in accordance with claim 16, wherein the said pathogenic microbe comprises at least one of fungus, bacterium, chlamydia, mycoplasma and virus.

18. The method for treating rhinosinusitis in accordance with claim 16, wherein the said inflammatory mediator comprises at least one of eosinophil, any component of eosinophil, IgE, IgG3, histamine, free radicals, excitotoxins, complement, cytokines, and prostaglandins.

19. An intra-sinonasal delivery device with a continual positive pressure and anti-backflow mechanism comprising:
A container holding a pharmaceutical composition;
at least one pair of air and liquid valves with a correct opposite flow directions to deliver the said pharmaceutical composition into the sinonasal cavities of a mammal through a nostril fitting wherein the pharmaceutical composition delivered into the sinonasal cavities of a mammal reduces the load of pathogenic microbes and inflammatory agents without causing adverse events comprising antibiotic resistance and disturbances to the body's immune function.

20. The intra-sinonasal delivery device in accordance to claim 19, wherein the said nostril fitting delivers the pharmaceutical composition into the sinonasal cavities of a mammal to remove a drug resistant microorganism from the sinonasal cavities of the said mammal.

21. The intra-sinonasal delivery device in accordance to claim 19, wherein the said nostril fitting delivers the pharmaceutical composition into the sinonasal cavities of a mammal to remove an inflammatory agent from the sinonasal cavities of the said mammal.

22. The intra-sinonasal delivery device in accordance to claim 19, wherein the said nostril fitting delivers the pharmaceutical composition into the sinonasal cavities of a mammal to remove an allergic agent from sinonasal cavities of the said mammal.

23. A collecting kit for collecting the out-flow liquid from the sinonasal cavities of a mammal comprising:
a sinonasal delivery device with a continual positive pressure and anti-backflow mechanism;
the said sinonasal delivery device further comprising a first container holding a first liquid for delivery into the sinonasal cavities and at least one first nostril fitting for delivering the said first liquid into the nasal cavities;
a second container for collecting a second liquid wherein the second liquid is the out-flow of the first liquid after the said first liquid is delivered into the sinonasal cavities of the mammal and the second container comprises a funnel-cylinder like member or a second nostril fitting for collecting the said second liquid;

24. The collecting kit in accordance with claim 23, wherein the second liquid collected in the second container contains the nasal wash specimen from the sinonasal cavities of a mammal.

25. The collecting kit in accordance with claim 23, wherein the second liquid collected in the second container contains the out-flow drug after the said drug is administered into the sinonasal cavities of a mammal by using any intranasal delivery method.

26. The collecting kit in accordance with claim 23, wherein the second liquid collected in the second container is used to monitor the efficacy of vaccination.
